(19) United States
(12) Patent Application Publication
(10) Pub. No.: US 2022/0220530 A1
(43) Pub. Date: Jul. 14, 2022

(54) Detection of Glycosaminoglycans

(71) Applicant: Baebies, Inc., Durham, NC (US)

(72) Inventors: Melissa Langer, Durham, NC (US); Rajendra Singh, Durham, NC (US)

(73) Assignee: Baebies, Inc., Durham, NC (US)

(21) Appl. No.: 17/613,147

(22) PCT Filed: May 26, 2020
(86) PCT No.: PCT/US2020/034545

§ 371 Date: Nov. 22, 2021

Related U.S. Application Data
(60) Provisional application No. 62/851,908, filed on May 23, 2019.

(51) Int. Cl. C12Q 1/37; G01N 33/58

(52) U.S. Cl. CPC C12Q 1/37; G01N 2400/40; G01N 33/582

(57) ABSTRACT

A method of detecting glycosaminoglycans in a sample, the method including: providing a sample potentially including glycosaminoglycans; combining with the sample: an enzyme, an inhibitor modulated by the presence of glycosaminoglycans; and a labeled substrate cleavable by the enzyme, wherein the labeled substrate comprises a label that is released when cleaved by the enzyme; detecting the released label and thereby inferring the presence, absence or quantity of glycosaminoglycans, wherein the released label is inversely proportional to the presence of glycosaminoglycans in the sample.

### RELATED APPLICATIONS

This application claims priority to and the benefit of U.S. Patent App. No. 62/851,908, filed on May 23, 2019.

### BACKGROUND OF THE INVENTION

Glycosaminoglycans (GAGs) are heteropolysaccharides composed of repeating disaccharide units. Aberrant catabolism of glycosaminoglycans (GAGs) with consequent intralysosomal accumulation of the undegraded products causes a group of lysosomal storage disorders collectively known as mucopolysaccharidoses (MPSs). MPSs are recognized by increased excretion in urine of partially degraded GAGs which ultimately result in progressive cell, tissue, and organ dysfunction. There are twelve different enzymes involved in the stepwise degradation of GAGs. Deficiencies in each of those enzymes result in different MPSs, all sharing a series of clinical features, though variable degrees characterized by the accumulation of different GAGs. Usually MPSs are characterized by a chronic and progressive course, with different degrees of severity.

Traditionally, MPSs are assayed by analysis of urinary GAGs. Several methods have been devised, such as dye binding and mass spectrometry methods. Existing methods suffer from a variety of shortcomings, including complex sample preparation, lack of sensitivity, lengthy processes, and in some cases, expensive instrumentation. There is a need in the art for methods for MPS screening applications that simplify sample preparation, have adequate sensitivity, and are quick and cost effective.

### SUMMARY OF THE INVENTION

The invention provides a method of detecting glycosaminoglycans in a sample. The method includes providing a sample potentially including glycosaminoglycans. The method includes; combining with the sample: an enzyme; an inhibitor modulated by the presence of glycosaminoglycans; and a labeled substrate cleavable by the enzyme, wherein the labeled substrate includes a label that is released when cleaved by the enzyme. The method includes detecting the released label and thereby inferring the presence, absence or quantity of glycosaminoglycans, wherein the released label is inversely proportional to the presence of glycosaminoglycans in the sample. In one aspect, step b includes combining the sample with the inhibitor prior to combining the sample with the enzyme. In another aspect, step b includes combining the sample with the inhibitor at substantially the same time that the sample is combined with the enzyme.

In some cases, the enzyme includes a hydrolase. In some cases, the enzyme includes a protease or peptidase. In some cases, the enzyme includes a serine protease.

In some cases, the enzyme includes a metalloproteinase. In some cases, the enzyme includes a caspase. In some cases, the enzyme includes an enzyme involved in a blood coagulation pathway. In some cases, the enzyme includes an enzyme involved in a fibrinolytic pathway. In some cases, the enzyme includes an enzyme involved in a thrombolytic pathway. In some cases, the enzyme is selected from the group consisting of: Factor II/IIa, Factor X/Xa, Factor V/Va, Factor VIII/VIIIa and modified versions of any of the foregoing. In some cases, the inhibitor is a protease or esterase inhibitor. In some cases, the inhibitor is a serpin. The enzyme may include modified versions or derivatives of the foregoing that retain an amount of the enzymatic activity of the native version sufficient for performing the assays of the invention.

In some cases, the inhibitor is selected from the group consisting of: heparin cofactor II, antithrombin III, Protein C and alpha 2 antiplasminogen inhibitor, and modified versions of any of the foregoing. In some cases, the inhibitor includes Antithrombin III and the enzyme includes Factor IIa. In some cases, the inhibitor includes Antithrombin and the enzyme includes Factor Xa. In some cases, the inhibitor includes Heparin cofactor II and the enzyme includes Factor IIa. In some cases, the inhibitor includes Protein C and the enzyme includes Factor Va and/or Factor VIIIa. In some cases, the inhibitor includes Alpha 2-antiplasmin and the enzyme includes Plasmin and/or Urokinase. In some cases, the inhibitor includes Plasminogen activator inhibitor 1 and the enzyme includes Tissue Plasminogen (tPA) and/or Urokinase (uPA). In some cases, the inhibitor includes Plasminogen activator inhibitor 1 and the enzyme includes Tissue Plasminogen (tPA) and/or Urokinase (uPA). The inhibitor may include modified versions or derivatives of the foregoing that retain an amount of the inhibition activity of the native version sufficient for performing the assays of the invention.

In some cases, the labeled substrate is cleavable by a protease. In some cases, the labeled substrate is cleavable by a hydrolase. In some cases, the labeled substrate is cleavable by a protease or peptidase. In some cases, the enzyme includes a serine protease. In some cases, the labeled substrate is cleavable by a metalloproteinase. In some cases, the labeled substrate is cleavable by a caspase. In some cases, the labeled substrate is cleavable by an enzyme involved in a blood coagulation pathway. In some cases, the labeled substrate is cleavable by an enzyme involved in a fibrinolytic pathway. In some cases, the labeled substrate is cleavable by an enzyme involved in a thrombolytic pathway. In some cases, the labeled substrate is cleavable by an enzyme is selected from the group consisting of: Factor II/IIa, Factor X/Xa, Factor V/Va, or Factor VIII/VIIIa.

In some cases, the labeled substrate has a formula: [substrate]-[cleavage site]-[label]; wherein: [substrate] includes a peptide or fragment from the zymogen form of the enzyme that is activated upon cleavage; [cleavage site] includes a hydrolytically cleavable bond; [label] includes a detectable label.

In some cases, the detectable label includes a fluorescent label (i.e., a label that is fluorescent upon cleavage from the substrate), chemiluminescent label, bioluminescent label, chromophore label, or mass tag. In some cases, the detectable label includes a fluorescent label selected from the group consisting of coumarins, naphthalene sulfonamides, acridines, acridones, xanthenes, fluoresceins, rhodamines, oxazines, resorufins and cyanines. In some cases, the detectable label includes a chemiluminescent label selected from the group consisting of acridinium esters, dioxetanes and luminol derivatives. In some cases, the detectable label includes a bioluminescent label selected from the group consisting of coelenterazines and luciferins. In some cases, the detectable label includes a mass tag having a molecular weight ranging from about 100 Da to about 2000 Da. Various exemplary structures are set forth in the detailed description below.

In some cases, the label is selected from the group consisting of: fluorescent labels, chemiluminescent labels, bioluminescent, chromophore and mass labels. In some cases, the label includes a chromophore moiety. In some embodiments, the label is a coumarin derivative.

In some cases, the sample is selected from the group consisting of: reconstituted dried blood spot samples; plasma; serum; blood; urine; synovial fluid, bone and cartilage tissue. In some cases, the sample includes a reconstituted dried blood spot. In some cases, the sample consists of a reconstituted dried blood spot having an area of less than about 10 mm². In some cases, the sample consists of a reconstituted dried blood spot having an area of less than about 9 mm². In some cases, the sample consists of a reconstituted dried blood spot having an area of about 8 mm². In some cases, the sample consists of a reconstituted dried blood spot, the dried blood spot composed of dried blood produced from fresh blood in a quantity ranging from about 1 μL to about 10 μL. In some cases, the sample consists of a reconstituted dried blood spot, the dried blood spot composed of dried blood produced from fresh blood in a quantity ranging from about 2 μL to about 7 μL. In some cases, the sample consists of a reconstituted dried blood spot, the dried blood spot composed of dried blood produced from fresh blood in a quantity ranging from about 2.7 μL to about 3.4 μL. In some cases, the sample is from a fetus or newborn infant.

The invention includes a method of diagnosing a mucopolysaccharidosis. The method includes using the methods described herein for detecting glycosaminoglycans in a sample; and in subjects from the set exhibiting elevated glycosaminoglycans relative to normal samples, testing for a panel of mucopolysaccharidoses. In one aspect, panel may include one or more tests for a set of conditions selected from: MPS I, MPS II, MPS III A, MPS III B, MPS III C, MPS III D, MPS IVA, MPS IV B, MPS VI and MPS VII. In one aspect, the panel includes tests for deficiencies of enzymes selected from the group consisting of: α-iduronidase; α-iduronide sulfatase; N-Acetyl α-glucosaminidase; N-sulfoglucosamine sulfohydrolase; α-glucosaminide-N-acetyl transferase; N-acetylglucosamine 6-sulfatase; N-acetylgalactosamine 6-sulfatase; β-galactosidase; N-acetylgalactosamine-4-sulfatase; and β-glucuronidase.

The invention also provides a kit having reagents for conducting the methods of the invention. In one case, the kit includes packaging materials. The packaging materials store an enzyme, an inhibitor modulated by the presence of glycosaminoglycans; and a labeled substrate cleavable by the enzyme, wherein the labeled substrate includes a label that is released when cleaved by an enzyme. In some cases, the kit includes a control having glycosaminoglycans. The control may include glycosaminoglycans at different concentrations. In some cases, the kit includes a calibrator having a set of glycosaminoglycans solutions at differing concentrations covering a predetermined dynamic range. In some cases, the kit includes software to analyse data for screening for the presence of glycosaminoglycans in the sample. In some cases, the kit includes a blood spot collection card. The kit may include instructions for using the reagents together with a sample for detecting the released label and thereby inferring the presence, absence or quantity of glycosaminoglycans, wherein the released label is inversely proportional to the presence of glycosaminoglycans in the sample.

The invention also includes a method of screening a set of subjects for a mucopolysaccharidosis. The method includes providing the kit of the invention; collecting samples from the set of subjects; testing the samples using the reagents from the kit; identifying a subset of the set of subjects having.

The invention provides a method of diagnosing a mucopolysaccharidosis. The method includes performing a set of enzyme activity assays on subjects identified in step d as having elevated glycosaminoglycans relative to normal samples. In some cases, the mucopolysaccharidosis is selected from the group consisting of: MPS I, MPS II, MPS III A, MPS III B, MPS III C, MPS III D, MPS IVA, MPS IV B, MPS VI and MPS VII. In some cases, the mucopolysaccharidosis is caused by a deficiency of an enzyme selected from the group consisting of: α-iduronidase; α-iduronide sulfatase; N-Acetyl α-glucosaminidase; N-sulfoglucosamine sulfohydrolase; α-glucosaminide-N-acetyl transferase; N-acetylglucosamine 6-sulfatase; N-acetylgalactosamine 6-sulfatase; β-galactosidase; N-acetylgalactosamine-4-sulfatase; and β-glucuronidase. In some cases, elevated glycosaminoglycans are at least about 15 ng/mL glycosaminoglycans or higher. In some cases, elevated glycosaminoglycans are at least about 20 ng/mL glycosaminoglycans or higher.

### BRIEF DESCRIPTION OF DRAWINGS

FIG. 1A and FIG. 1B illustrate schematic diagrams of an assay protocol for detection of GAG in a sample;

FIG. 2 illustrates a flow diagram of an example of the method of FIG. 2 of measuring GAGs in a DBS sample using an enzyme inhibition assay;

FIG. 3A is a plot showing a standard curve for dermatan sulfate in a buffer on a microtiter plate assay format;

FIG. 3B is a plot showing a standard curve for heparan sulfate in a buffer on a microtiter plate assay format;

FIG. 3C is a plot showing a standard curve for keratan sulfate in a buffer on a microtiter plate assay format; and

FIG. 4 is a plot showing modulation of Factor II activity by heparan sulfate extracted from a DBS sample at different concentrations.

FIG. 5 is a table and a plot showing modulation of Factor II (FII) activity by GAGs in a DBS extract using the coupled assay format;

FIG. 6 is a table and a plot showing modulation of Factor II (FII) activity by GAG in a DBS extract using an endpoint assay format; and

FIG. 7 is a table and a plot showing modulation of Factor II (FII) activity by heparan sulfate using the uncoupled assay format.

### DETAILED DESCRIPTION OF THE INVENTION

The invention uses the modulation of enzymatic activity by a GAG in the presence of an enzyme, an inhibitor and a labeled substrate of the enzyme which serves as a reporter of the enzymatic activity. The invention provides methods and kits that make use of an enzyme, an inhibitor and a labeled substrate to measure modulation of enzymatic activity by a GAG. The modulation of enzymatic activity is correlated with the GAG concentration. GAGs modulate the activity of an enzyme (e.g., a protease) either directly, for example, cleaving a zymogen or interacting with an inhibitor of the enzyme.

Enzymatic Deficiencies

The invention is useful for screening for enzymatic deficiencies. “Enzymatic deficiency” means that activity from the enzyme is either reduced relative to a normal range or is missing altogether from the sample. The invention is useful for screening subjects for enzymatic deficiencies associated with the accumulation of GAGs.

Examples of disorders associated with the accumulation of GAGs include mucopolysaccharidoses. Examples of mucopolysaccharidoses include MPS I, MPS II, MPS III A, MPS III B, MPS III C, MPS III D, MPS IVA, MPS IV B, MPS VI, MPS VII, and MPS IX. Further examples include enzymatic deficiencies associated with the following enzymes.

Examples enzymes which, when deficient, are associated with the accumulation of GAGs include α-iduronidase; α-iduronide sulfatase; N-Acetyl α-glucosaminidase; N-sulfoglucosamine sulfohydrolase; α-glucosaminide-N-acetyl transferase; N-acetylglucosamine 6-sulfatase; N-acetylgalactosamine 6-sulfatase; β-galactosidase; N-acetylgalactosamine-4-sulfatase; and β-glucuronidase.

Samples

Any sample potentially accumulating GAGs may be used. Examples include blood, blood products, plasma, serum, dried blood extracts, and urine. Samples are preferably from humans but may be other animals as well.

In a preferred embodiment, the sample is a reconstituted dried blood spot (DBS). DBS may be collected on a variety of substrates, such as paper cards made from cellulose. Blood may be collected, e.g., via venipuncture or skin puncture. Cards for collecting DBS are commercially available, e.g., Whatman 903 from Tisch Scientific Co. (Cleves, Ohio), Ahlstrom 266 (Perkin Elmer, Waltham, Mass.). Spots may be air dried in a suitable location selected to minimize or avoid contamination of the blood spots. DBS may be stored on the card in a gas-impermeable bag, optionally including a desiccant. DBS may be stored in a freezer with a temperature of −20° C. or lower as soon as possible following drying. DBS specimens may ideally be transported at low temperatures, e.g., using dry ice.

DBSs may be reconstituted by punching out a spot from the card. In one embodiment, the amount of DBS required for the assay is from about 6 mm² to about 10 mm², or from about 7 mm² to about 9 mm², or about 8 mm², which is the area of one 3.2 mm diameter DBS punch. In a preferred embodiment, a single 3.2 mm diameter DBS punch is used. However, it will be appreciated that two or more punches can be combined for a single extraction.

In one embodiment, the quantity of blood required for the assay is from about 1 μL to about 10 μL, or from about 2 μL to about 7 μL, or from about 2.7 μL to about 3.4 μL, or about 3.1 μL, which is the amount of blood on one 3.2 mm diameter DBS punch.

In one embodiment, a single 3.2-mm diameter DBS punch is extracted in 100 μL of extraction solution (100 mM Tris, 100 mM ammonium chloride, 0.1% (v/v) Tween 20, pH 7.5; Sigma) with shaking at 600 rpm for 2 hours at 37° C.

Other examples of reagent for reconstituting dried blood spots include:
100 mM Tris, 150 mM NaCl, 20 mM CaCl2), pH 8.0
0.1% Tween 20
0.1% Tween 20+150 mM NaCl

Extraction solution volumes may preferably be in the range of from about 25 μL to about 125 μL, or from about 50 μL to about 100 μL, or about μL, or about 100 μL.

Extraction time may preferably range from about 15 min to about 2.5 hours, or from about 30 min to about 2 hours, or about 30 min, or about 1 hours or about 2 hours.

Extraction temperature may preferably be ambient or about 37° C.

Number of DBS punches may preferably be from 1, 2 or 3; preferably 2; more preferably 1.

Enzyme

The invention makes use of an enzyme capable of cleaving the substrate (described in more detail below). The enzyme is modulated by an inhibitor, and the inhibitor is in turn modulated by the presence of GAGs.

Examples of suitable enzymes include caspases, esterases, hydrolases such as peptidases and proteases, hydrolytic enzymes involved in the blood coagulation and fibrinolytic and thrombolytic pathways, proteases, metalloproteinases, serine proteases, and derivatives, analogs or modified versions of the foregoing that retain some or all of the activity of the native enzyme.

In one embodiment, the enzyme is a coagulation factor. In one embodiment, the enzyme is selected from Factor II/IIa, Factor X/Xa, Factor V/Va, Factor VIII/VIIIa, and derivatives, analogs or modified versions of the foregoing that retain some or all of the activity of the native enzyme.

Derivatives, analogs or modified versions of enzymes useful in the invention will retain sufficient activity and other characteristics, such as ability to be inhibited by inhibitors, to effectuate the methods of the invention. Such activity can be determined experimentally by one of skill in the art.

The enzyme may be from any species, such as human, mouse, etc. The enzyme may have a native amino acid sequence or may have a modified, non-native sequence, which retains some portion of all of the native enzymatic activity. The enzyme may include artificial amino acids or other chemical modifications. For example, in one embodiment, a modified enzyme retains 70%, 80%, 90%, 95%, 99% or has enhanced activity relative to the native enzyme under the same conditions.

Inhibitor

The invention makes use of an inhibitor selected to modulate cleavage of the substrate by the enzyme. The inhibitor itself is modulated by the presence of GAGs. For example, the presence of GAGs in the reaction may increase inhibition of the enzyme by the inhibitor.

Examples of serine protease inhibitors include serpins such as antithrombin and antitrypsin. In one embodiment, the inhibitor is an antithrombin, such as human antithrombin, and/or a derivative, analog or modified version thereof that retains activity of antithrombin, including its ability to be inhibited by GAGs.

Derivatives, analogs or modified versions of inhibitors useful in the invention will retain inhibitory and other characteristics sufficient to effectuate the methods of the invention. Such activity can be determined experimentally by one of skill in the art.

The inhibitor may be from any species, such as human, mouse, etc. Typically, the inhibitor and enzyme will be from the same species. The inhibitor may have a native amino acid sequence or may have a modified, non-native sequence, which retains some portion of all of the native inhibition. The enzyme may include artificial amino acids or other chemical modifications. For example, in one embodiment, a modified inhibitor retains 70%, 80%, 90%, 95%, 99% or has enhanced activity relative to the native inhibitor under the same conditions.

Examples of suitable inhibitor-enzyme pairs include:
antithrombin III, factor IIa
antithrombin, factor Xa
heparin cofactor II, factor IIa
protein C, factor Va and/or factor VIIIa
alpha-2-antiplasmin, plasmin
plasminogen activator inhibitor 1, tissue type plasminogen activator (tPA) and/or urokinase (uPA)
plasminogen activator inhibitor 2, tissue type plasminogen activator (tPA) and/or urokinase (uPA)

Other inhibitor-enzyme pairs will be recognizable by those of skill in the art in view of this specification.

Labeled Substrate

The invention makes use of a labeled substrate cleavable by the enzyme.

The labeled substrate has the following general structure:
[substrate]-[cleavage site]-[label]

The [substrate], [cleavage site], and [label] are selected to permit cleavage by the enzyme.

For example, the [substrate] may be a peptide or fragment from the zymogen form of the enzyme that is activated upon cleavage, such peptide residues can include natural and unnatural amino acids.

For example, the [cleavage site] may be an amide, carbamate or ester bond that is hydrolytically cleaved.

For example, the [label] may be a fluorescent, chemiluminescent, bioluminescent, mass tag or chromophore or other label.

Examples of suitable fluorescent labels include coumarins, acridines, acridones, napthalene sulfonamides, xanthenes, fluoresceins, rhodamines, oxazines, resoruf ins and cyanines.

Examples of suitable chemiluminescent labels include acridinium esters, dioxetanes and luminol derivatives.

Examples of bioluminescent labels include coelenterazines and luciferins.

Examples of suitable mass labels include cleaved moieties of molecular weight between 100-2000 daltons.

In one example, a Factor IIa (Thrombin) substrate with an AMC label has the following structure:

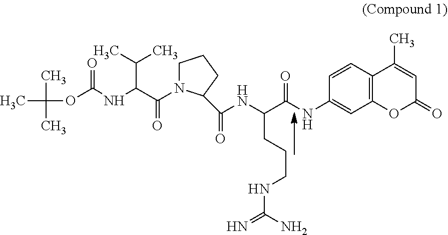 [SMILES: Cc1cc(=O)oc2cc(NC(=O)C(CCCNC(=N)N)NC(=O)C3CCCN3C(=O)C(NC(=O)OC(C)(C)C)C(C)C)ccc12]

In another example, a Factor Xa substrate with an AMC label has the following structure:

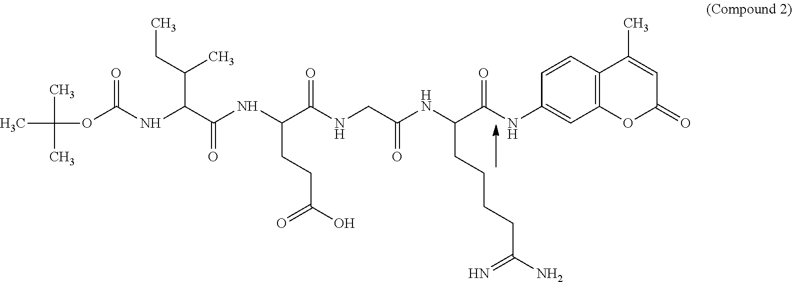 [SMILES: CCC(C)C(NC(=O)OC(C)(C)C)C(=O)NC(CCC(=O)O)C(=O)NCC(=O)NC(CCCCC(=N)N)C(=O)Nc1ccc2c(C)cc(=O)oc2c1]

In one example, a Factor IIa (Thrombin) substrate with an ANSN label has the following structure:

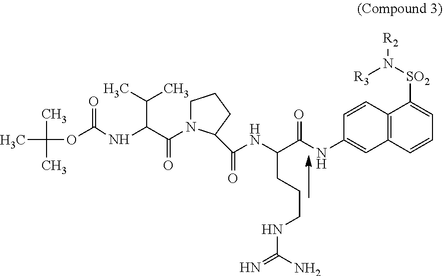

where R2 is H, alkyl (C1-C20), cycloaklyl (C4-C20), aryl (C6-C20) and combinations thereof and R3 is H, alkyl (C1-C20), cycloaklyl (C4-20), aryl (C6-C20) and combinations thereof: R2 and R3 together could be part of a cycloalkyl group.

In one example, a Factor IIa (Thrombin) substrate with an HMRG label has the following structure:

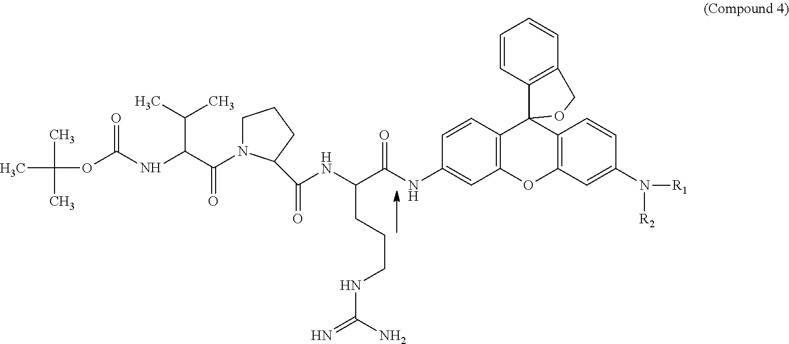

where R1 is H, alkyl (C1-C20), cycloaklyl (C4-C20), aryl (C6-C20) and combinations thereof and R2 is H, alkyl (C1-C20), cycloaklyl (C4-20), aryl (C6-C20) and combinations thereof: R1 and R2 together could be part of a cycloalkyl group.

In one example, a Factor IIa (Thrombin) substrate with a luciferin label has the following structure:

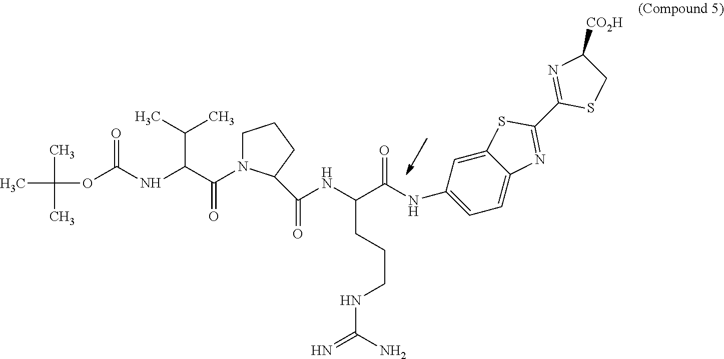 [SMILES: CC(C)C(NC(=O)OC(C)(C)C)C(=O)N1CCCC1C(=O)NC(CCCNC(=N)N)C(=O)Nc1ccc2nc(C3=N[C@@H](C(=O)O)CS3)sc2c1]

In another embodiment, the substrate is a 6-amino-1-naphthalenesulfonamide-based (ANSN) fluorogenic substrate cleaved by FXa. This substrate has the structure:

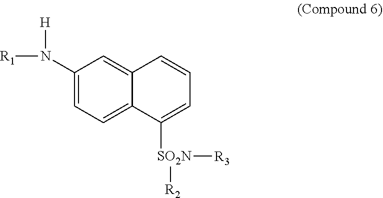

where R1 is a tripeptide of which the COOH-terminal residue is typically an arginine; and R2 and R3 may be a hydrogen, alkyl, aryl, or cycloalkyl group. These substrates are commercially available from Haematologic Technologies, Inc. (Essex Junction, Vermont). Examples include:
D-AFK-ANSNH-iC₄H₉.2HBr, for plasmin
Mes-D-LGR-ANSN(C₂H₅)₂, for factor Xa
D-LPR-ANSNH-C₃H₇.2 HCl, for factor XIa
D-LPR-ANSNH-C₆H₁₁.2 HCl, for thrombin
BOC-D-VLR-ANSNH-C₄H₉, for aPC

### Methods of the Invention

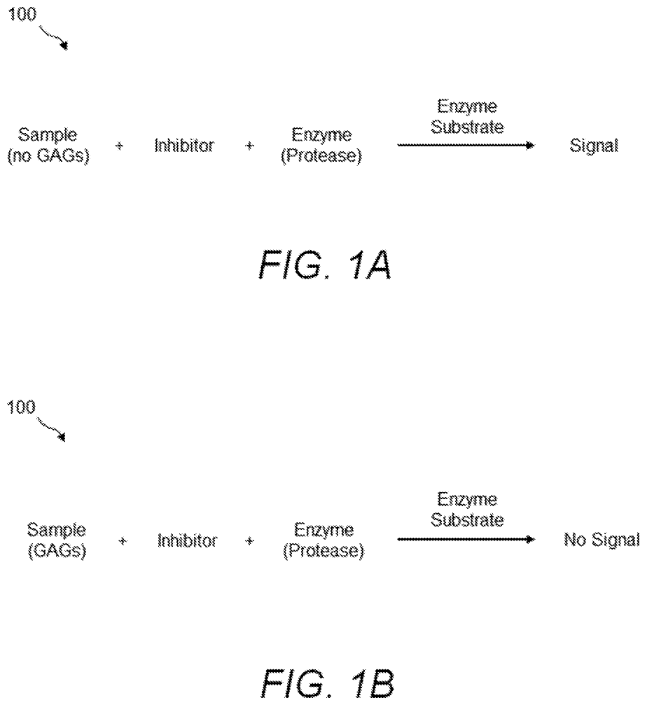

FIG. 1A and FIG. 1B illustrate schematic diagrams of an example of an inhibition assay protocol 100 for detection of GAG-modulated enzyme activity in a sample. Assay protocol 100 uses an enzyme (e.g., a serine protease), an inhibitor of the enzyme (e.g., a serine protease inhibitor), and a labeled substrate of the enzyme (e.g., a fluorogenic substrate) to determine the level of GAG(s) in a sample. Briefly, when GAG is bound to an inhibitor, the inhibition of the enzyme by the inhibitor is significantly increased. The presence of GAG and/or the amount of GAG in a sample is then determined based on the generation of a detectable signal that is correlated to the level of enzyme activity.

Referring now to FIG. 1A, an inhibition assay includes combining a sample (e.g., extracts from a DBS sample), an enzyme inhibitor that is regulated by GAG, and an enzyme. A labeled enzyme substrate is then added to the sample/inhibitor/enzyme reaction and the generation of a detectable signal is determined. In the absence of GAG in the sample (“no GAGs”), the interaction between the inhibitor and enzyme is minimal and a detectable signal is produced.

Referring now to FIG. 1B, a labeled enzyme substrate is added to a sample/inhibitor/enzyme reaction and the generation of a detectable signal is determined. In the presence of GAG in the sample (“GAGs”), the GAG in the sample binds to the inhibitor and the interaction between the inhibitor and enzyme is significantly increased. Since the enzyme is bound by the GAG-inhibitor, cleavage of the labeled substrate is blocked and a lower (or no) detectable signal is produced. The greater the GAG concentration in the sample, the lower the detectable signal produced (i.e., higher [GAG]=lower signal).

The format of the inhibition assay may be varied by changing the order in which the assay components (i.e., enzyme, enzyme inhibitor and sample) are added.

In one embodiment, the addition of the enzyme and enzyme inhibitor are “coupled”, wherein the enzyme reagent (e.g., serine protease) and inhibitor reagent (e.g., serine protease inhibitor) are incubated together for a period of time prior to the addition of the sample.

The enzyme reagent and inhibitor reagent may, for example, be incubated together for about 10 min, or about 30 min, or about 1 hour, or about 2 hours prior to the addition of the sample.

Incubation temperature may be ambient or about 37□.

In one embodiment, the addition of the enzyme and enzyme inhibitor are “uncoupled”, wherein the enzyme inhibitor reagent (e.g., serine protease inhibitor) and the sample are incubated together for a period of time prior to the addition of the enzyme reagent (e.g., serine protease).

The enzyme inhibitor reagent and sample may, for example, be incubated together for about 10 min, or about 30 min, or about 1 hour, or about 2 hours prior to the addition of the enzyme reagent.

Incubation temperature may be ambient or about 37□.

In one embodiment, the inhibition assay is a kinetic assay, wherein a detection signal is read at intervals, typically once per minute, over a period of time. For example, in one embodiment, the RFU slope is measured at one minute time intervals from from 0 to 10 minutes.

In one embodiment, the inhibition assay is an endpoint assay, wherein the enzyme-substrate reaction is stopped after a sufficient period of time and a detection signal is read, e.g., about 30 min.

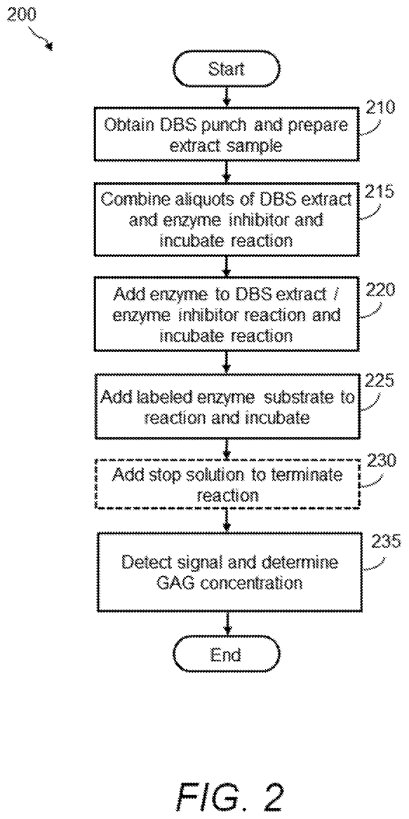

FIG. 2 illustrates a flow diagram of an example of a method 200 of measuring GAGs in a DBS sample using an enzyme inhibition assay. In this example, the addition of the enzyme and enzyme inhibitor are uncoupled, wherein the enzyme inhibitor reagent and sample are incubated together for a period of time prior to the addition of the enzyme. Method 200 includes, but is not limited to, the following steps.

Preparing Sample from Dried Blood Spot

At a step 210, a DBS punch is obtained and a sample extract is prepared. For example, one DBS punch may be incubated in 100 μL of Extraction Solution (for example, 100 mM Tris, 150 mM NaCl, 20 mM CaCl2), 0.1% Tween 20, pH 8.0) for 1 hour at 37 C on a plate shaker set to 600 rpm.

Combining Dried Blood Spot Extract with Enzyme Inhibitor

At a step 215, aliquots of the DBS extract and an enzyme inhibitor are combined and incubated for a period of time sufficient for binding of GAGs to the inhibitor. In one example, the enzyme inhibitor is a serpin such as antithrombin III (ATM). In one example, the aliquot provided is in the range of 5-15 μL.

The inhibitor may be included in a suitable buffer solution. For example, a suitable buffer is 100 mM Tris, 150 mM NaCl, 0.1 mg/mL BSA, pH 8.0.

The inhibitor may be provided at a concentration and volume selected to ensure that the protease will produce a signal that is not completely flattened by the inhibitor when GAGs are not present. For example, the enzyme may be provided at a concentration between about 8 and about 250 nM and a volume ranging from about 5 μL to about 15 μL. Preferably, the inhibitor is added in an amount which is at least about 2× the amount of enzyme, or at least about 2.5× the amount of enzyme, or at least about 3× the amount of enzyme, or at about 3× the amount of enzyme.

Adding Enzyme

At step 220, an enzyme is added to the DBS extract/enzyme inhibitor reaction and incubated for a period of time. In one example, the enzyme is a serine protease such as factor II (FII).

The enzyme may be provided in a buffer. For example, a suitable buffer is a Tris buffer having a pH in the range of about 7 to about 8.5. For example, the Tris buffer may be 100 mM Tris, 150 mM NaCl, 20 mM CaCl2), pH 8.0. As another example the Tris buffer may be 10 mM Tris, 40 mM NaCl, 0.25 mg/mL PEG, pH 7.5.

The enzyme may be provided at a concentration and volume selected to ensure that the protease will produce a signal that is not completely flattened by the inhibitor when GAGs are not present. For example, the enzyme may be provided at a concentration between about 2 and about 64 nM and a volume ranging from about 5 μL to about 15 μL.

Adding Enzyme Substrate

At a step 225, an enzyme substrate is added to the DBS extract/inhibitor/enzyme reaction to monitor the activity of the enzyme in the reaction. In one example, the enzyme substrate is a fluorogenic substrate that liberates a fluorescent dye upon cleavage, such as an aminomethyl coumarin protease substrate.

The enzyme substrate may be provided in a buffer. For example, a suitable buffer is . . .

For example, the enzyme substrate may be provided at a concentration between about 20 μM and about 200 μM and a volume ranging from about 20 μL to about 20 μL. The total amount of substrate per reaction preferably ranges from about 2.5 nmol to about 7.5 nmol, or from about 4 nmol to about 6 nmol, or is about 5 nmol.

Adding Stop Solution (Optional)

At an optional step 230, a stop solution is added (optional) to terminate the enzyme-substrate reaction. For example, in a stop solution may be added to the enzyme-substrate reaction to monitor an endpoint of the reaction. An example of a suitable stop solution is 2% (w/v) citric acid. In another embodiment, the assay has a kinetic readout, and a stop solution is not required. A suitable amount is added to stop the reaction, e.g., 40 uL (˜70% of the final reaction volume).

Detecting Signal

At a step 235, a signal is detected and the amount of GAG in a sample is determined. In one example, the detection mode is an endpoint detection mode. Signal detection may be accomplished using a sensor selected for the specific signal produced by the label. For example, in one example, a signal is detected using a Biotek HTX plate reader with Gen5 software. One of skill in the art may select suitable excitation and emission wavelengths depending on the label used.

In another example, the detection mode is a kinetic mode. In one embodiment, as soon as kinetic detections begin as soon as substrate is added. Detections are automatically taken every minute for the duration of the reaction.

In one embodiment of method 200 of FIG. 2, the addition of the enzyme and enzyme inhibitor are coupled, wherein the enzyme reagent and inhibitor reagent are incubated together for a period of time prior to the addition of the sample. For example, at step 215 an enzyme/enzyme inhibitor reagent is prepared and aliquoted into wells of a microtiter plate. At step 220, an aliquot of the DBS extract is added to the enzyme/enzyme inhibitor reagent in the wells of the microtiter plate. Method 200 then continues.

### Examples

Detecting GAGs Using a Fluorescence-Based Enzyme Activity

To demonstrate the feasibility of detecting GAGs using a fluorescence-based enzyme activity assay, we performed microtiter plate assays for dermatan sulfate, heparan sulfate, and keratan sulfate.

Standard curves for each GAG were prepared by diluting the GAGs to particular concentrations in assay buffer (100 mM Tris, 150 mM NaCl, 20 mM CaCl2), pH 8.0; all from Sigma Aldrich, St. Louis, Mo.) and performing two-fold serial dilutions for each GAG in assay buffer, as follows: 0 to 3097 ng/mL (0 to 64 nM) heparan sulfate; 0 to 484 ng/mL (0 to 10 nM) heparin sulfate; 0 to 23002 (0 to 64 nM) dermatan sulfate; 0 to 32258 ng/mL (0 to 1000 nM) keratan sulfate.

A reagent solution of the inhibitor Antithrombin III (ATIII; from Abcam (Cambridge, Mass.)) and the enzyme Factor II (FII; from R&D Systems (Minneapolis, Minn.) was prepared in assay buffer at final concentrations of 20 nM and 64 nM, respectively, and incubated for 10 minutes at room temperature.

Five (5) μL of the combined ATIII/FII reagent was pipetted into each well of a black half-area microtiter plate. After incubating the plate at 37° C. for 1 hour, 50 μL of FII substrate (Boc-Val-Pro-Arg-amido-4-methylcoumarin hydrochloride salt; (Bachem; Bubendorf, Switzerland)) diluted to 100 μM in assay buffer was added to each well and the plate was immediately read at 360 nm excitation/460 nm emission.

Kinetic reads were obtained every 5 minutes over the course of 30 min and the plate was incubated at 37□ between reads. Normalized slopes were calculated by setting the slope of 0 ng/mL GAG to 100% activity and calculating the relative percent activity for each level of GAG (normalized slope=calculated slope/[(slope for 0 ng/mL GAG/GAG/100)]. RFU=raw fluorescence units.

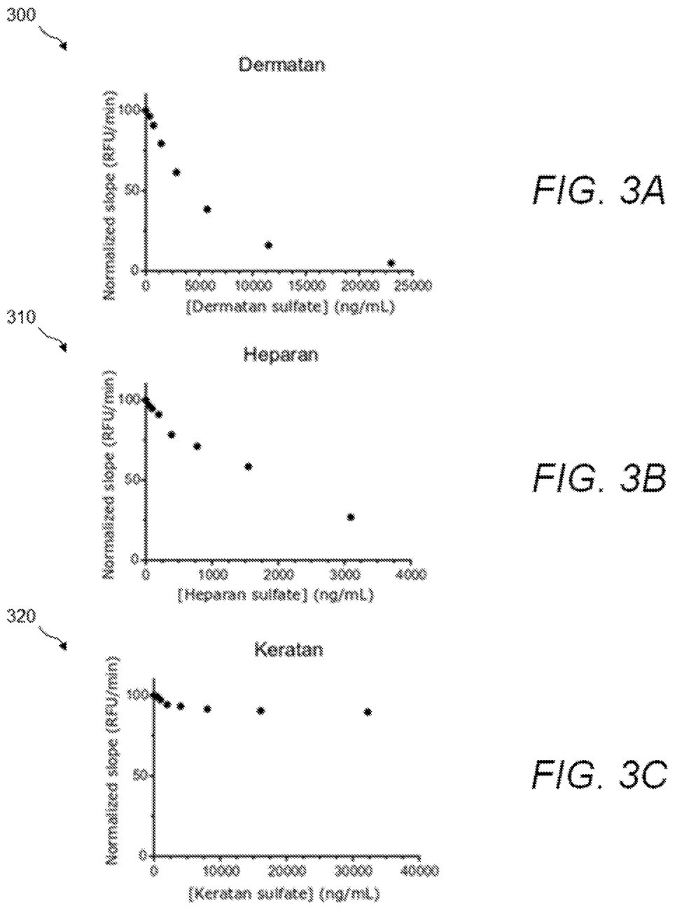

FIG. 3A is a plot 300 showing a standard curve for dermatan sulfate in a buffer on a microtiter plate assay format. The data show the standard curve is linear across the range of 0 nM to 65 nM for dermatan sulfate.

FIG. 3B is a plot 310 showing a standard curve for heparan sulfate in a buffer on a microtiter plate assay format. The data show the standard curve is linear across the range of 0 nM to 65 nM for heparan sulfate.

FIG. 3C is a plot 320 showing a standard curve for keratan sulfate in a buffer on a microtiter plate assay format. The data show the standard curve is linear across the range of 0 nM to 1,000 nM for keratan sulfate.

Demonstrating Proof of Principle of the GAG Assay from a Dried Blood Spot

To demonstrate proof of principle of the GAG assay from a dry blood spot (DBS) sample, DBS samples were prepared in-house using packed red blood cells (Tennessee Blood Services (Memphis, Tenn.) and heparan sulfate as follows: packed red blood cells were washed with saline and combined with a volume of heat-inactivated serum (Seracare Technologies (Milford, Mass.)) to a final hematocrit of about 50%. A heparin sulfate (HS) standard curve was prepared in saline. Small volumes of the standard curve were diluted into blood so that the final concentrations were 0-2000 ng/mL. This corresponds to 3.1 uL of standard curve volume for every 100 uL of blood. 100 uL is the volume of a dry blood spot and 3.1 uL is the volume of sample that is expected to be in a single punch. After mixing, 100 μL of each spiked blood sample was spotted onto filter paper (GE 903) and allowed to dry overnight. Dried spots were stored in a ziplock bag with desiccant at −80° C. until use.

A single 3.2-mm diameter DBS punch at each HS concentration (0, 80, 160, 400, 800, 1600 and 2000 ng/mL) was extracted in 100 μL of extraction solution (100 mM Tris, 100 mM ammonium chloride, 0.1% (v/v) Tween 20, pH 7.5; Sigma) with shaking at 600 rpm for 2 hours at 37° C.

Five (5) μL of each DBS-HS extract was incubated with 54 human heparan cofactor II (Haematologic Technologies (Essex Junction, Vt.)) diluted to 70 nM in assay buffer (10 mM Tris, 40 mM sodium chloride, 0.25 mg/mL PEG-8000, pH 7.5) in a black half-area microtiter plate. After a 1 hour incubation at room temperature, 54 of Factor II diluted to 6 nM in assay buffer was added to each well and the plate was incubated for 30 minutes at room temperature. Forty (40) μL of a 30 μM FII substrate was added to each well and the plate was immediately read at 360 nm excitation/460 nm emission. Kinetic reads were obtained by reading the plate every minute for 30 minutes. The plate remained in the plate reader set at room temperature for the entire 30-minute duration.

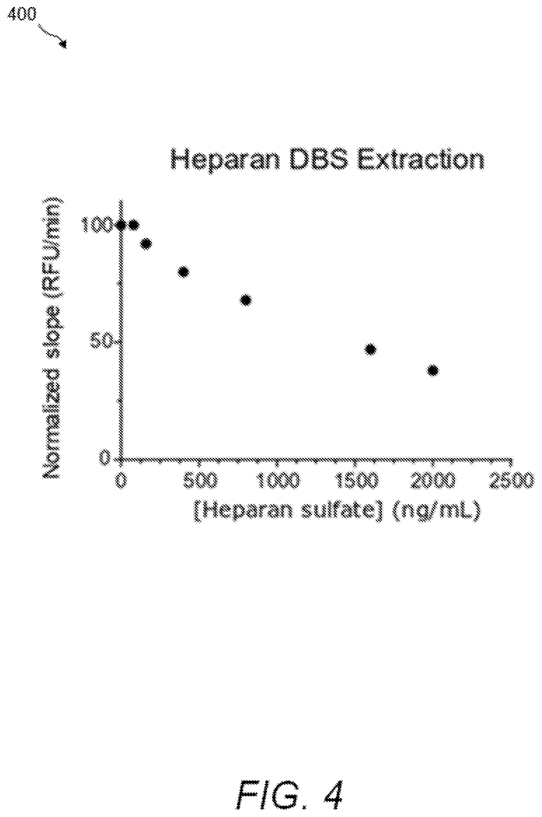

FIG. 4 is a plot 400 showing modulation of Factor II activity by heparan sulfate extracted from a DBS sample at different concentrations. The data show a linear correlation between heparan sulfate concentration and normalized slope. The raw data with % CV over 3 replicates is shown below in Table 1.

TABLE 1
HS concentrations

| HS (ng/mL) | Mean ± St. Dev | % CV |
|---|---|---|
| 0 | 100 ± 1.3 | 1.3% |
| 80 | 100 ± 0.9 | 0.9% |
| 160 | 92 ± 1.1 | 1.2% |
| 400 | 80 ± 1.7 | 2.1% |
| 800 | 68 ± 0.8 | 1.2% |
| 1600 | 47 ± 0.9 | 1.9% |
| 2000 | 38 ± 1.1 | 2.9% |

Assay with Combined Protease and Inhibitor Stock

To demonstrate proof of principle of the coupled inhibition assay format, an enzyme/enzyme inhibitor reagent stock was prepared and incubated prior to the addition of sample.

We performed the following steps:
Incubate 1 DBS punch in 100 uL of Extraction Solution (100 mM Tris, 150 mM NaCl, 20 mM CaCl2), 0.1% Tween 20, pH 8.0) for 1 h at 37° C.
Dilute FII from R&D Systems to 64 nM in Assay Buffer (100 mM Tris, 150 mM NaCl, 20 mM CaCl2), pH 8.0)
Dilute ATIII from Abcam to 250 nM in Assay Buffer (100 mM Tris, 150 mM NaCl, 20 mM CaCl2), pH 8.0)
Dilute FII substrate (Boc-Val-Pro-Arg-amido-4-methylcoumarin substrate from Bachem) to 100 uM in Assay Buffer (100 mM Tris, 150 mM NaCl, 20 mM CaCl₂), pH 8.0)
Combine 1 part 64 nM FII with 1 part 250 nM ATIII in a 0.6-mL microcentrifuge tube and incubate at ambient conditions for 10 min
Combine 5 uL of DBS extract with 5 uL of the ATIII/FII stock in a black half-area 96 well microtiter plate
Incubate the plate at 37° C. for 1 h
Add 50 uL of 100 uM FII substrate to each well
Take kinetic fluorescence readings of the plate every minute for 30 minutes using a Biotek HTX plate reader with Gen5 software; excitation at 360 nm and emission at 460 nm
Calculate the slope for each sample from 0 to 10 min

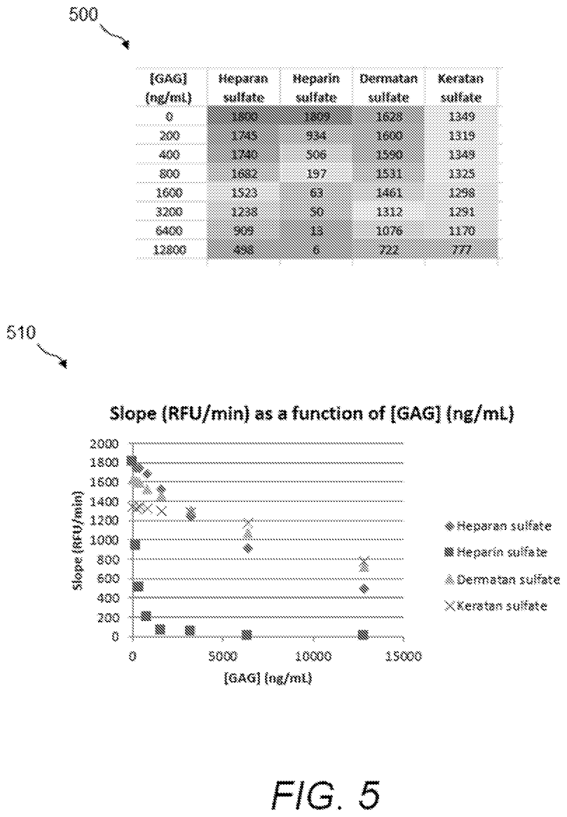

FIG. 5 is a table 500 and a plot 510 showing modulation of Factor II (FII) activity by GAGs in the DBS extract using the coupled assay format. The data show that the assay format with combined protease and inhibitor stock demonstrated a dose-dependent response for all four GAGs (i.e., heparan sulfate, heparin sulfate, dermatan sulfate, and keratan sulfate) across the concentrations tested. Heparin sulfate exhibited the most inhibition while keratan sulfate exhibited the least.

Assay with Endpoint Readout

To demonstrate proof of principle of an assay format that uses an endpoint readout, a stop solution is added to terminate the enzyme-substrate reaction at 30 min.

We performed the following steps:
Incubate 1 DBS punch in 100 uL of Extraction Solution (100 mM Tris, 150 mM NaCl, 20 mM CaCl2), 0.1% Tween 20, pH 8.0) for 1 h at 37° C.
Dilute FII from R&D Systems to 48 nM in Assay Buffer (100 mM Tris, 150 mM NaCl, 20 mM CaCl2), pH 8.0).
Dilute ATIII from Abcam to 250 nM in Assay Buffer (100 mM Tris, 150 mM NaCl, 20 mM CaCl2), pH 8.0).
Dilute FII substrate (Boc-Val-Pro-Arg-amido-4-methylcoumarin substrate from Bachem) to 100 uM in Assay Buffer (100 mM Tris, 150 mM NaCl, 20 mM CaCl2), pH 8.0).
Combine 1 part 32, 48, and 64 nM FII with 1 part 250 nM ATIII in 0.6-mL microcentrifuge tubes and incubate at ambient conditions for 10 min.
Combine 10 uL of DBS extract with 10 uL of the ATIII/FII stock in a black half-area 96 well microtiter plate.
Incubate the plate at 37° C. for 1 h.
Add 30 uL of 160 uM FII substrate to each well and immediately take a fluorescence reading of the plate at 360 nm EX/460 nm EM using a Biotek HTX plate reader with Gen5 software
Incubate the plate at ambient conditions for 30 minutes.
Add 40 uL of 2% citric acid to each well.
Measure the fluorescence of the plate at 360 nm EX/460 nm EM.

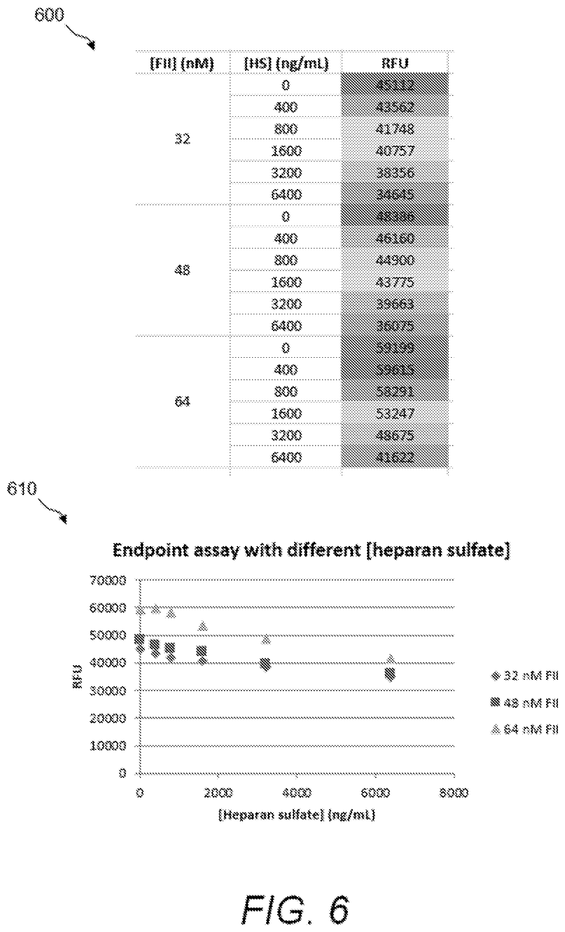

FIG. 6 is a table 600 and a plot 610 showing modulation of Factor II (FII) activity by GAG in a DBS extract using an endpoint assay format for the 48 nM FII. Note that this data was produced by diluting heparin sulfate in Extraction Solution.

The results are illustrated in FIG. 6. The data show that the endpoint assay format in which 2% citric acid is added to stop the reaction after 30 minutes of incubation with FII substrate demonstrated a dose-dependent response for heparan sulfate. Using 32 or 48 nM FII resulted in signal distinction between 0 and 400 ng/mL heparan sulfate while using 64 nM resulted in signal distinction between 0 and 800 ng/mL heparan sulfate.

Assay with Protease and Inhibitor Added Separately

To demonstrate proof of principle of the uncoupled inhibition assay format, an inhibitor/sample reaction was prepared and incubated prior to the addition of the enzyme.

In this example, “extracted” and “spiked” samples were used. The samples were prepared as follows: 1) extracted samples were prepared by adding heparan sulfate (HS) into QCBP blood and spotting aliquots of the HS-QCBP blood sample onto filter paper cards; and 2) “spiked” samples were prepared by spiking aliquots of HS into a QCBP blood extract.

We performed the following steps:
Incubate 1 DBS punch in 100 uL of Extraction Solution (100 mM Tris, 100 mM NH4Cl, 0.1% Tween 20, pH 7.5) on a shaker set to 600 rpm for for 2 h at 37° C.
Dilute Human Heparan Cofactor II (HHCoFII) to 70 nM in Assay Buffer (10 mM Tris, 40 mM NaCl, 0.25 mg/mL PEG-8000, pH 7.5).
Dilute FII to 6 nM in Assay Buffer (10 mM Tris, 40 mM NaCl, 0.25 mg/mL PEG-8000, pH 7.5.
Dilute FII substrate (Boc-Val-Pro-Arg-amido-4-methylcoumarin substrate from Bachem) to 30 uM in Assay Buffer (10 mM Tris, 40 mM NaCl, 0.25 mg/mL PEG-8000, pH 7.5).
Combine 10 uL of DBS extract with 10 uL of 70 nM HHCoFII in a black half-area microtiter plate.
Incubate the plate at ambient conditions for 1 h.
Add 10 uL of 6 nM FII to each well.
Incubate the plate at ambient conditions for 30 min.
Add 40 uL of 30 uM FII substrate to each well.
Take kinetic fluorescence readings of the plate at 360 nm EX/460 nm EM every minute for 30 minutes using a Biotek HTX plate reader with Gen5 software.
Calculate the slope for each sample from 0 to 10 min.

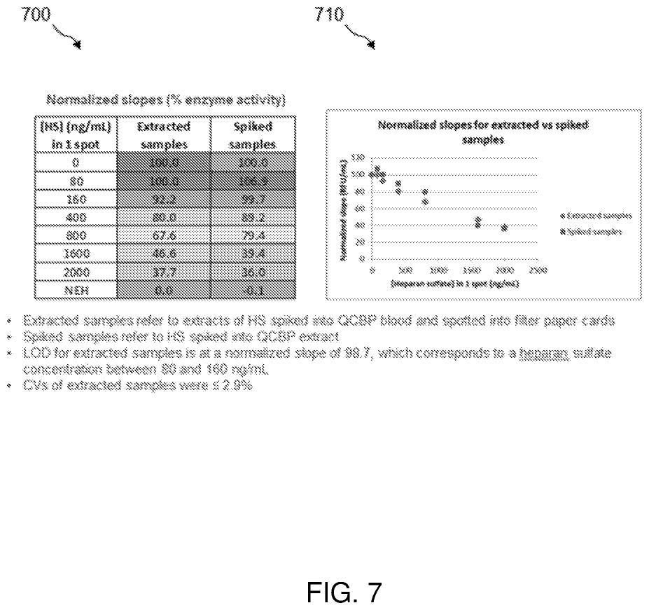

FIG. 7 is a table 700 and a plot 710 showing modulation of Factor II (FII) activity by heparan sulfate using the uncoupled assay format. The data show a distinction between 0 and 160 ng/mL of heparan sulfate was observed for both spiked and extracted samples of heparan that were incubated with HHCoFII prior to the addition of FIIa.

The results are illustrated in FIG. 7. Distinction between 0 and 160 ng/mL of heparan sulfate was observed for both spiked and extracted samples of heparan sulfate that were incubated with HHCoFII followed by FIIa.

The present invention has been disclosed in the above teachings with sufficient clarity and conciseness to enable one skilled in the art to make and use the invention, to know the best mode for carrying out the invention, and to distinguish it from other inventions and from what is old. Many variations and obvious adaptations will readily come to mind, and these are intended to be contained within the scope of the invention as claimed below.

We claim:

1. A method of detecting glycosaminoglycans in a sample, the method comprising:
a. providing a sample potentially including glycosaminoglycans;
b. combining with the sample:
i. an enzyme;
ii. an inhibitor modulated by the presence of glycosaminoglycans; and
iii. a labeled substrate cleavable by the enzyme, wherein the labeled substrate comprises a label that is released when cleaved by the enzyme;
c. detecting the released label and thereby inferring the presence, absence or quantity of glycosaminoglycans, wherein the released label is inversely proportional to the presence of glycosaminoglycans in the sample.

2. The method of claim 1 wherein step b comprises combining the sample with the inhibitor prior to combining the sample with the enzyme.

3. The method of claim 1 or 2 wherein the enzyme comprises a hydrolase.

4. The method of claim 1 or 2 wherein the enzyme comprises a protease or peptidase.

5. The method of claim 1 or 2 wherein the enzyme comprises a serine protease.

6. The method of claim 1 or 2 wherein the enzyme comprises a metalloproteinase.

7. The method of claim 1 or 2 wherein the enzyme comprises a caspase.

8. The method of claim 1 or 2 wherein the enzyme comprises an enzyme involved in a blood coagulation pathway.

9. The method of claim 1 or 2 wherein the enzyme comprises an enzyme involved in a fibrinolytic pathway.

10. The method of claim 1 or 2 wherein the enzyme comprises an enzyme involved in a thrombolytic pathway.

11. The method of claim 1 or 2 wherein the enzyme is selected from the group consisting of: Factor II/IIa, Factor X/Xa, Factor V/Va, Factor VIII/VIIIa and modified versions of any of the foregoing.

12. The method of claim 1 or 2 wherein the inhibitor is a protease or esterase inhibitor.

13. The method of claim 1 or 2 wherein the inhibitor is a serpin.

14. The method of claim 1 or 2 wherein the inhibitor is selected from the group consisting of: heparin cofactor II, antithrombin III, Protein C and alpha 2 antiplasminogen inhibitor, and modified versions of any of the foregoing.

15. The method of claim 1 or 2 wherein:
a. the inhibitor comprises antithrombin III and/or a derivative, analog or modified version thereof; and
b. the enzyme comprises factor IIa and/or a derivative, analog or modified version thereof.

16. The method of claim 1 or 2 wherein:
a. the inhibitor comprises an antithrombin and/or a derivative, analog or modified version thereof; and
b. the enzyme comprises factor Xa and/or a derivative, analog or modified version thereof.

17. The method of claim 1 or 2 wherein:
a. the inhibitor comprises heparin cofactor II and/or a derivative, analog or modified version thereof; and
b. the enzyme comprises factor IIa and/or a derivative, analog or modified version thereof.

18. The method of claim 1 or 2 wherein:
a. the inhibitor comprises protein C and/or a derivative, analog or modified version thereof; and
b. the enzyme comprises Factor Va and/or Factor VIIIa and/or a derivative, analog or modified version thereof.

19. The method of claim 1 or 2 wherein:
a. the inhibitor comprises alpha-2-antiplasmin and/or a derivative, analog or modified version thereof; and
b. the enzyme comprises plasmin and/or a derivative, analog or modified version thereof.

20. The method of claim 1 or 2 wherein:
a. the inhibitor comprises plasminogen activator inhibitor 1 and/or a derivative, analog or modified version thereof; and
b. the enzyme comprises tissue tissue type plasminogen activator (tPA) and/or urokinase (uPA) and/or a derivative, analog or modified version thereof.

21. The method of claim 1 or 2 wherein:
a. the inhibitor comprises plasminogen activator inhibitor 2; and
b. the enzyme comprises tissue type plasminogen activator (tPA) and/or urokinase (uPA) and/or a derivative, analog or modified version thereof.

22. The method of claim 1 or 2 wherein the labeled substrate is cleavable by a hydrolase.

23. The method of claim 1 or 2 wherein the labeled substrate is cleavable by a protease or peptidase.

24. The method of claim 1 or 2 wherein the labeled substrate is cleavable by a serine protease.

25. The method of claim 1 or 2 wherein the labeled substrate is cleavable by a metalloproteinase.

26. The method of claim 1 or 2 wherein the labeled substrate is cleavable by a caspase.

27. The method of claim 1 or 2 wherein the labeled substrate is cleavable by an enzyme involved in a blood coagulation pathway.

28. The method of claim 1 or 2 wherein the labeled substrate is cleavable by an enzyme involved in a fibrinolytic pathway.

29. The method of claim 1 or 2 wherein the labeled substrate is cleavable by an enzyme involved in a thrombolytic pathway.

30. The method of claim 1 or 2 wherein the labeled substrate is cleavable by an enzyme selected from the group consisting of: Factor II/IIa, Factor X/Xa, Factor V/Va, or Factor VIII/VIIIa.

31. The method of claim 1 or 2 wherein the labeled substrate has a formula: [substrate]-[cleavage site]-[label]; wherein:
a. [substrate] comprises a peptide or fragment from the zymogen form of the enzyme that is activated upon cleavage;
b. [cleavage site] comprises a hydrolytically cleavable bond; and
c. [label] comprises a detectable label.

32. The method of claim 31 wherein the detectable label comprises a fluorescent label, chemiluminescent label, bioluminescent label, chromophore label, or mass tag.

33. The method of claim 31 wherein the detectable label comprises a fluorescent label selected from the group consisting of coumarins, napthalene sulfonamides, acridines, acridones, xanthenes, fluoresceins, rhodamines, oxazines, resorufins and cyanines.

34. The method of claim 31 wherein the detectable label comprises a chemiluminescent label selected from the group consisting of acridinium esters, dioxetanes and luminol derivatives.

35. The method of claim 31 wherein the detectable label comprises a bioluminescent label selected from the group consisting of coelenterazines and luciferins.

36. The method of claim 31 wherein the detectable label comprises a mass tag having a molecular weight ranging from about 100 Da to about 2000 Da.

37. The method of claim 1 or 2 wherein the substrate has the following structure:

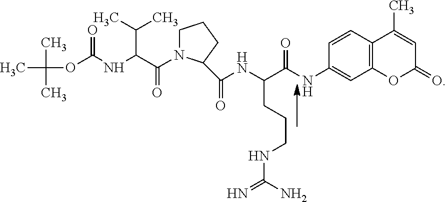 [SMILES: Cc1cc(=O)oc2cc(NC(=O)C(CCCNC(=N)N)NC(=O)C3CCCN3C(=O)C(NC(=O)OC(C)(C)C)C(C)C)ccc12]

38. The method of claim 1 or 2 wherein the substrate has the following structure:

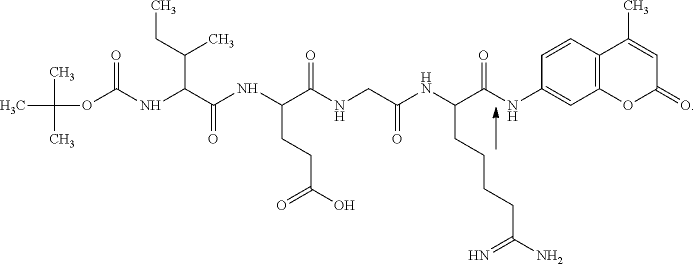 [SMILES: CCC(C)C(NC(=O)OC(C)(C)C)C(=O)NC(CCC(=O)O)C(=O)NCC(=O)NC(CCCCC(=N)N)C(=O)Nc1ccc2c(C)cc(=O)oc2c1]

39. The method of claim 1 or 2 wherein the substrate has the following structure: 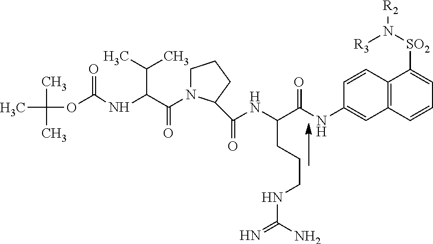
where R2 is H, alkyl (C1-C20), cycloaklyl (C4-C20), aryl (C6-C20) and combinations thereof and R3 is H, alkyl (C1-C20), cycloaklyl (C4-20), aryl (C6-C20) and combinations thereof; wherein R2 and R3 together may optionally be part of a cycloalkyl group.

40. The method of claim 1 or 2 wherein the substrate has the following structure: 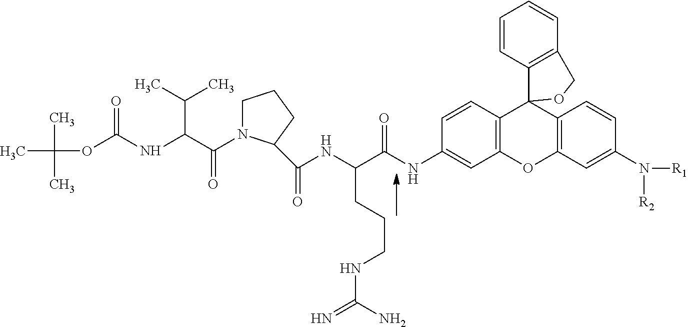
where R1 is H, alkyl (C1-C20), cycloaklyl (C4-C20), aryl (C6-C20) and combinations thereof and R2 is H, alkyl (C1-C20), cycloaklyl (C4-20), aryl (C6-C20) and combinations thereof; wherein R1 and R2 together may optionally be part of a cycloalkyl group.

41. The method of claim 1 or 2 wherein the substrate has the following structure:

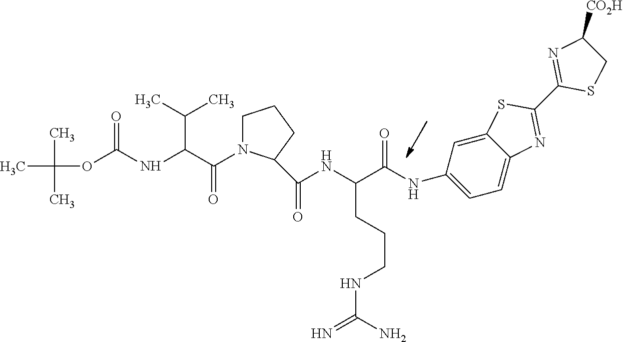 [SMILES: CC(C)C(NC(=O)OC(C)(C)C)C(=O)N1CCCC1C(=O)NC(CCCNC(=N)N)C(=O)Nc1ccc2nc(C3=N[C@@H](C(=O)O)CS3)sc2c1]

42. The method of claim 1 or 2 wherein the label is selected from the group consisting of: fluorescent labels, chemiluminescent labels, bioluminescent labels, chromophore labels and mass labels.

43. The method of claim 1 or 2 wherein the label comprises a chromophore moiety.

44. The method of claim 1 or 2 where the label is a coumarin derivative.

45. The method of any of the foregoing claims wherein the sample is selected from the group consisting of: reconstituted dried blood spot samples; plasma; serum; blood; urine; synovial fluid, bone and cartilage tissue.

46. The method of any of claims 1-44 wherein the sample comprises a dried blood spot extract.

47. The method claim 46 wherein the sample consists of a dried blood spot extract having an area of less than about 10 mm².

48. The method claim 46 wherein the sample consists of a dried blood spot extract having an area of less than about 9 mm².

49. The method claim 46 wherein the sample consists of a dried blood spot extract having an area of about 8 mm².

50. The method claim 46 wherein the sample consists of a dried blood spot extract, wherein the dried blood is produced from fresh blood in a quantity ranging from about 1 μL to about 10 μL.

51. The method claim 46 wherein the sample consists of a dried blood spot extract, wherein the dried blood is produced from fresh blood in a quantity ranging from about 2 μL to about 7 μL.

52. The method claim 46 wherein the sample consists of a dried blood spot extract, wherein the dried blood is composed of dried blood produced from fresh blood in a quantity ranging from about 2.7 μL to about 3.4 μL.

53. The method of any of the foregoing claims wherein the sample is from a fetus or newborn infant.

54. A method of diagnosing a mucopolysaccharidosis comprising:
a. using the method of any of the foregoing claims to screen a set of subjects to identify subjects having glycosaminoglycans that are elevated relative to a threshold level; and
b. in subjects from the set exhibiting elevated glycosaminoglycans, testing for a panel of mucopolysaccharidoses.

55. The method of claim 54 wherein the panel includes tests for a set of conditions selected from the group consisting of: MPS I, MPS II, MPS III A, MPS III B, MPS III C, MPS III D, MPS IVA, MPS IV B, MPS VI and MPS VII.

56. The method of claim 54 wherein the panel includes tests for deficiencies of enzymes selected from the group consisting of: α-iduronidase; α-iduronide sulfatase; N-Acetyl α-glucosaminidase; N-sulfoglucosamine sulfohydrolase; α-glucosaminide-N-acetyl transferase; N-acetylglucosamine 6-sulfatase; N-acetylgalactosamine 6-sulfatase; β-galactosidase; N-acetylgalactosamine-4-sulfatase; and β-glucuronidase.

57. A kit comprising reagents selected for conducting the methods of any of claim 1 and following.

58. A kit comprising packaging materials comprising the following reagents:
a. an enzyme,
b. an inhibitor modulated by the presence of glycosaminoglycans; and
c. a labeled substrate cleavable by the enzyme, wherein the labeled substrate comprises a label that is released when cleaved by the serine protease.

59. The kit of claim 57 or 58 further comprising a control comprising glycosaminoglycans.

60. The kit of claim 57 or 58 further comprising a calibrator comprising a set of glycosaminoglycans solutions at differing concentrations covering a predetermined dynamic range.

61. The kit of claim 57 or 58 further comprising software to analyse data for screening for the presence of glycosaminoglycans in the sample.

62. The kit of claim 57 or 58 further comprising a blood spot collection card.

63. A method of screening a set of subjects for a mucopolysaccharidosis, the method comprising:
a. providing the kit of any of claims 57-62;
b. collecting samples from the set of subjects;
c. testing the samples using the reagents from the kit;
d. identifying a subset of the set of subjects having elevated glycosaminoglycans relative to normal.

64. A method of diagnosing a mucopolysaccharidosis, the method comprising performing the method of claim 63 followed by performing a set of enzyme activity assays on subjects identified in step d as having elevated glycosaminoglycans.

65. The method of claim 64 wherein the mucopolysaccharidosis is selected from the group consisting of: MPS I, MPS II, MPS III A, MPS III B, MPS III C, MPS III D, MPS IVA, MPS IV B, MPS VI and MPS VII.

66. The method of claim 64 wherein the mucopolysaccharidosis is caused by a deficiency of an enzyme selected from the group consisting of: α-iduronidase; α-iduronide sulfatase; N-Acetyl α-glucosaminidase; N-sulfoglucosamine sulfohydrolase; α-glucosaminide-N-acetyl transferase; N-acetylglucosamine 6-sulfatase; N-acetylgalactosamine 6-sulfatase; β-galactosidase; N-acetylgalactosamine-4-sulfatase; and β-glucuronidase.
